(19) United States
(12) Patent Application Publication
(10) Pub. No.: US 2008/0255499 A1
(43) Pub. Date: Oct. 16, 2008

(54) Compositions for Peritoneal Dialysis

(75) Inventor: Bum-Seok Kim, Seoul (KR)

Correspondence Address: LUCAS & MERCANTI, LLP, 475 PARK AVENUE SOUTH, 15TH FLOOR, NEW YORK, NY, 10016, US

(73) Assignee: RenoBiz Co ., Ltd, Seoul (KR)

(21) Appl. No.: 12/093,873

(22) PCT Filed: Nov. 17, 2006
(86) PCT No.: PCT/KR2006/004855

§ 371 Date: Jun. 13, 2008

(30) Foreign Application Priority Data
Nov. 18, 2005 (KR) 10-2005-0110658

(51) Int. Cl. A61M 1/28; C07C 229/00; A61K 31/195; A61K 31/7004; A61P 3/12

(52) U.S. Cl. 604/28; 562/553; 514/561; 514/23

(57) ABSTRACT

Disclosed herein are a composition for peritoneal dialysis comprising an α-keto amino acid, and a method for peritoneal dialysis using the same. The composition allows peritoneal dialysis to be effected without the problems accompanying conventional compositions, including tissue toxicity and uremia.

### TECHNICAL FIELD

The present invention relates to a composition for peritoneal dialysis comprising α-keto amino acid and to a method of conducting peritoneal dialysis using the same.

### BACKGROUND ART

Renal failure is described as the inability of the kidneys to excrete wastes and to help maintain the electrolyte balance. With the development of renal failure, the blood level of uremic substances, such as blood urea nitrogen (BUN), creatine (Cr), phosphorus (P), potassium (K) and organic acids, decreases, incurring various syndromes including hypertension, metabolic acidosis, hyperkalemia, and anemia, as well as fatigability, breathlessness, reduction of urine output, edema, and anorexia, which lead to death if left untreated. Currently, hemodialysis, peritoneal dialysis and kidney transplantation are the treatments for uremia.

Dialysis is well known as a type of renal replacement therapy used to provide an artificial replacement for kidney function that is lost due to renal failure. Dialysis may be effected outside or inside the body. In peritoneal dialysis, which is effected inside the body, solutes and water are exchanged via the peritoneum against a hypertonic solution. Peritoneal dialysis may be largely classified into intermittent peritoneal dialysis (IPD) and continuous ambulatory peritoneal dialysis (CAPD). CAPD, also having the advantage over IPD, features a long dwell time of the perfusion solution introduced into the peritoneal cavity, thereby allowing the exchange to be repeated about four times during the day.

Generally, the dialysate used in peritoneal dialysis comprises sodium, potassium, chlorine, calcium, magnesium, lactic acid and glucose. The pH of the dialysis solution is set in the range from about 5.0 to 5.9. This glucose-enriched, lactate buffered, low-pH dialysate, one of the most popular solutions for peritoneal dialysis, uses glucose as an osmotic agent, so that an appropriate osmotic gradient is formed across the peritoneum to conduct ultrafiltration, thereby allowing an excess of body fluids to migrate from the blood into the solution for peritoneal dialysis. Due to the high glucose level, low pH, high osmotic concentration and the formation of glucose degradation products (GDPs), however, the dialysate suffers from various problems. For example, the conventional dialysate is found to be highly non-biocompatible, as assayed through in vitro and animal experiments. In addition, it has been reported that the conventional dialysate causes peritoneal fibrosis, induces the formation of inflammatory cytokines and vascular endothelial growth factor (VEGF), and destroys the defense mechanism of the peritoneum. Particularly, glucose degradation products (GPDs) are derived upon heat sterilization of high glucose dialysis solution. Generally, GPDs comprise acetaldehyde, formaldehyde, methylgycoxal, glycoxal, 5-hydroxymethyl furaldehyde, 2-furaldehyde and 3-deoxyglucosone and, along with a high concentration of glucose, are known to accelerate the formation of advanced glycosylation end-products (AGEs) in the peritoneum and blood, which are associated with peritoneal fibrosis and systemic inflammatory response. In human tests, peritoneal injury was observed to gradually increase with increases in exposure of glucose to the peritoneum, which plays a causative role in the failure of peritoneal dialysis. Further, absorbed glucose may result in metabolic disorders, such as obesity, hyperglycemia, hyperlipidemia, etc.

In order to overcome the problems with glucose-enriched solutions for peritoneal dialysis, glucose-substitutable osmotic agents have been developed. Studies have revealed that amino acids can be used as osmotic agents. Amino acids are well absorbed and thus can be used as protein sources that are effective for nutrition-deficient patients. Also, a 1.1% amino acid dialysis solution realizes ultrafiltration similar to a 1.5% glucose dialysis solution. However, one of the main functions of dialysis is to reduce the high level of urea in the blood of patients with severe renal failure. The administration of amino acids, particularly at a dose of 100 mg/kg/day or higher, significantly increases blood nitrogen levels, aggravating the blood urea level. Therefore, amino acid dialysis solutions suffer from the disadvantage of requiring the very careful observation of blood urea levels during the application thereof.

Also, the risk of metabolic acidosis forces amino acid dialysis solutions to be used only in one out of every four rounds of dialysis, making it difficult to employ amino acid dialysis solutions throughout the dialysis process. Hence, non-amino acid dialysis solutions must inevitably be used, and the concomitant side effects thereof must be borne.

Leading to the present invention, intensive and thorough research into safe dialysis solutions, conducted by the present inventor, resulted in the finding that α-keto amino acid dialysates for peritoneal dialysis are safe, not showing the side effects of glucose or amino acid solutions.

### DISCLOSURE OF THE INVENTION

It is therefore an object of the present invention to provide a composition for peritoneal dialysis, comprising an α-keto amino acid.

It is another object of the present invention to provide a method for peritoneal dialysis using the composition.

### BRIEF DESCRIPTION OF THE DRAWINGS

FIG. 1 is a graph in which the optical densities of Neutril are plotted against dilution ratios.

FIG. 2 is a graph in which the optical densities of the composition for peritoneal dialysis of the present invention are plotted against dilution ratios.

FIG. 3 shows the absorption spectra of Neutril and the composition for peritoneal dialysis of the present invention.

FIG. 4 is a graph in which blood glucose levels are plotted against dialysis time in mice administered with a glucose lysate, an amino acid lysate, and an α-keto amino acid lysate.

FIG. 5 is a graph in which BUN levels are plotted against dialysis time in mice administered with a glucose lysate, an amino acid lysate, and an α-keto amino acid lysate.

FIG. 6 is a graph in which blood creatinine levels are plotted against dialysis time in mice administered with a glucose lysate, an amino acid lysate, and an α-keto amino acid lysate.

FIG. 7 is a graph in which blood protein levels are plotted against dialysis time in mice administered with a glucose lysate, an amino acid lysate, and an α-keto amino acid lysate.

FIG. 8 is a graph in which blood albumin levels are plotted against dialysis time in mice administered with a glucose lysate, an amino acid lysate, and an α-keto amino acid lysate.

FIG. 9 is a graph in which hsCRP (high sensitivity C-reactive protein) levels are plotted against dialysis time in mice administered with a glucose lysate, an amino acid lysate, and an α-keto amino acid lysate.

### BEST MODE FOR CARRYING OUT THE INVENTION

In accordance with an aspect, the present invention pertains to a composition for peritoneal dialysis, comprising an α-keto amino acid.

The terms, “α-keto amino acid”, as used herein, means an amino acid residue in which the α-amino moiety is substituted with an α-ketone moiety. Examples of the substituted amino acids useful in the present invention include glycine, alanine, serine, cysteine, aspartic acid, glutamine, glutamic acid, leucine, isoleucine, lysine, hydroxylysine, asparagine, tyrosine, tryptophan, histidine, phenylalanine, cystine, proline, hydroxyproline, threonine, methionine, hydroxymethionine and valine, with preference for leucine, isoleucine, phenylalanine and valine. These α-keto amino acids may be used alone or in combination. An example of using a combination of α-keto amino acids is Ketosteril™, commercially available from Fresenius Kabi Deutschland GmbH. It contains leucine, isoleucine, phenylalanine and valine, all having an α-keto moiety instead of α-amino moiety, and is used in the present invention as an illustrative, non-limiting example.

In addition, the α-keto amino acids of the present invention can be prepared according to well-known methods. For example, they can be synthesized chemically or microbiologically, that is, via production from microbes. Commercially available α-keto amino acids, such as Ketosteril™, may also be alternatives.

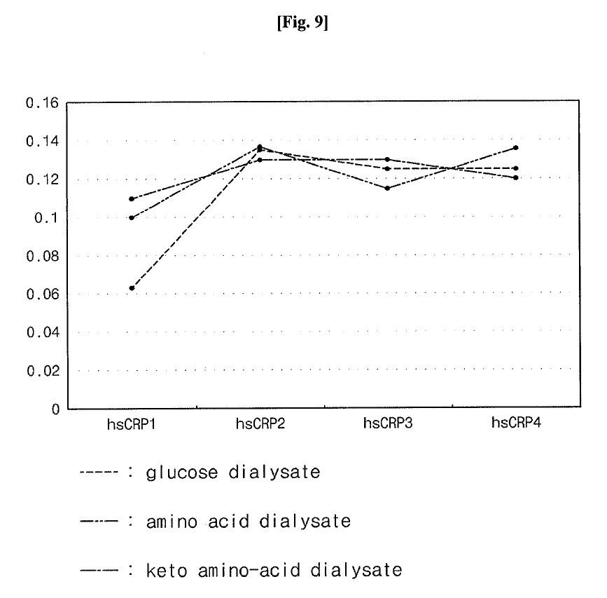

An α-keto amino acid solution for peritoneal dialysis in accordance with the present invention not only shows the advantages of conventionally used glucose dialysis solutions and amino acid dialysis solutions, but also overcomes the disadvantages of these dialysis solutions. For example, the composition of the present invention has the same dialysis capability and motility between semipermeable membranes as those of the commercially available dialysis solution Neutril™ (Baxter Healthcare SA, Singapore branch), as is apparent in the data of Tables 6 and 7, below. Free of glucose, the composition of the present invention eliminates the risk of side effects and toxicity attributable to glucose. In contrast to conventional amino acid solutions for peritoneal dialysis, the α-keto amino acid dialysate of the present invention does not increase BUN levels, and thus does not cause concomitant side effects, even when it is used in a large amount. Further, the dialysate for peritoneal dialysis in accordance with the present invention has an advantage over the conventional amino acid dialysis solutions in terms of protein provision for patients with renal failure. Thus, the dialysate of the present invention can effectively reduce protein-calorie malnutrition, which is a major predictor of morbidity and mortality in peritoneal dialysis patients with end-stage renal failure. Upon the application of the dialysate according to the present invention, α-keto amino acids are absorbed into the body and converted into standard amino acids, during which transaminases are stimulated to associate with the excess urine toxin BUN to form amino acids. Therefore, the dialysate according to the present invention considerably reduces BUN levels of patients with renal failure. Also, the oral administration of α-keto amino acids is reported to lead to the acceleration of protein synthesis and the suppression of protein degradation as well as the reduction of metabolic acidosis, attributable to sulfur-containing amino acids, which are mainly found in animal lipids. In this regard, it is reported that a conventional keto amino acid complex (Ketosteril™), when administered orally, can significantly reduce the metabolic acidosis of patients with chronic renal failure. Furthermore, the composition for peritoneal dialysis in accordance with the present invention can significantly reduce peritoneal fibrosis compared to glucose dialysis solutions (see FIG. 9).

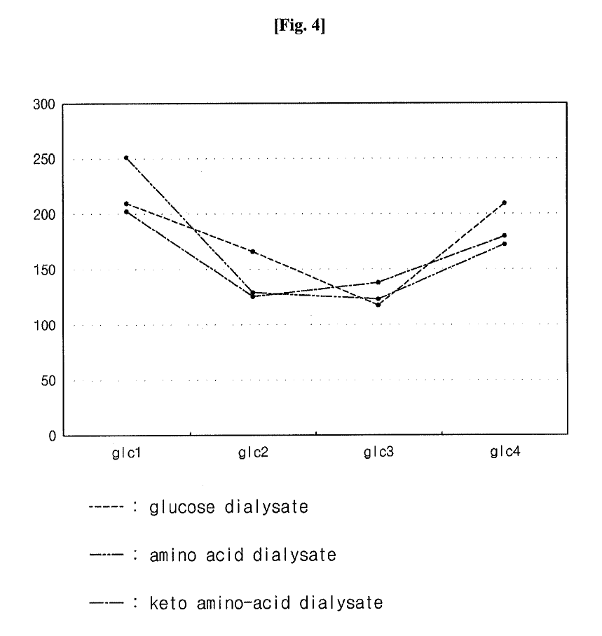

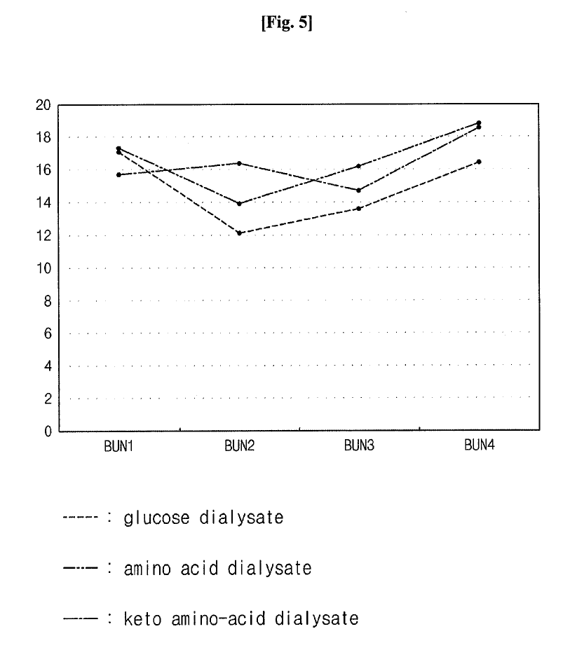

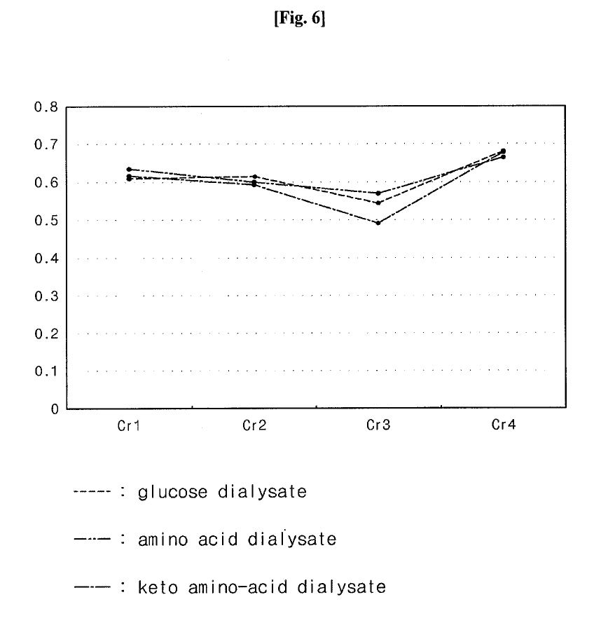

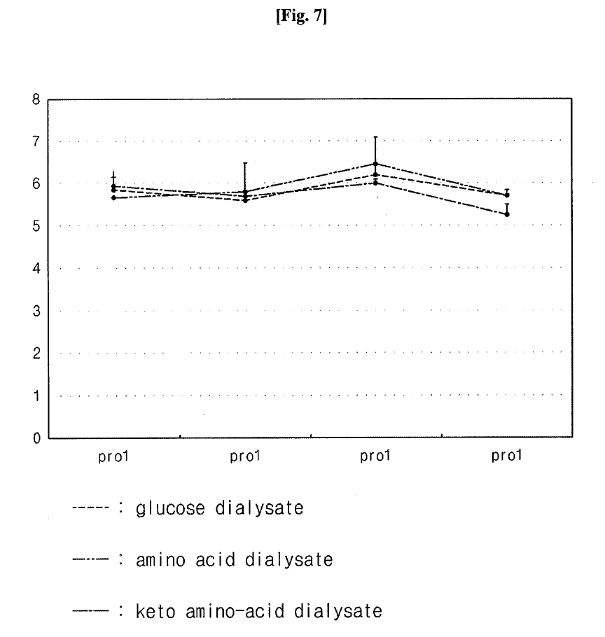

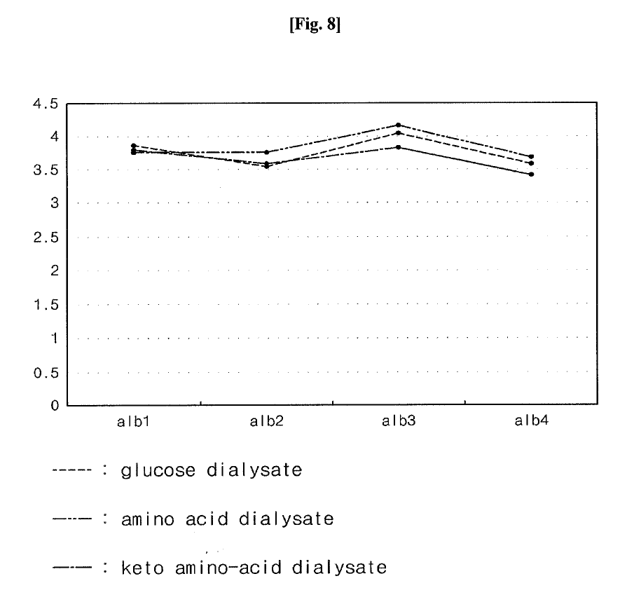

As elucidated above, the composition of the present invention can overcome the disadvantages of conventional glucose or amino acid dialysis solutions and can be effectively used as a dialysate for the peritoneal dialysis of patients with renal failure. Particularly, the composition for peritoneal dialysis in accordance with the present invention is found to be comparable to commercially available glucose solutions and amino acid solutions, in terms of safety and toxicity, as measured in safety and toxicity assays. Thus, the composition of the present invention can be safely used as a dialysate for peritoneal dialysis (see FIGS. 4 to 8).

The composition for peritoneal dialysis in accordance with the present invention comprises an α-keto amino acid in an amount from 8,000 to 40,000 mg/l, preferably in an amount from 10,000 to 37,000 mg/l and more preferably in an amount from 11,000 to 34,000 mg/l.

The composition comprising an α-keto amino acid in accordance with the present invention ranges in pH from 4.5 to 7.8, and preferably from 6.0 to 7.0.

In a preferred embodiment, the composition for peritoneal dialysis in accordance with the present invention may comprise a general dialysis solution in addition to α-keto acid. This general dialysis solution comprises an amino acid in which the α-amino moiety is not replaced by an α-amino moiety, glucose, an electrolyte or an organic acid salt. This amino acid is a general amino acid, examples of which include, but are not limited to, glycine, alanine, serine, cysteine, aspartic acid, glutamine, glutamic acid, leucine, isoleucine, lysine, hydroxylysine, asparagine, tyrosine, tryptophan, histidine, phenylalanine, cystine, proline, hydroxyproline, threonine, methionine, hydroxymethionine, and valine, with preference for lysine, threonine, tryptophan, histidine, tyrosine, and/or hydroxymethionine. In the total composition, the amount of the amino acid is on the order of 280 to 1200 mg/l, and preferably on the order of 340 to 1020 mg/l. The electrolyte may be exemplified by ion forms of sodium, potassium, chlorine, calcium, and/or magnesium, but are not limited thereto. Preferably, the composition comprises sodium in an amount from 125 to 140 mmol/L, potassium in an amount from 0 to 4 mmol/L, a chloride ion in an amount from 95 to 115 mmol/L, calcium in an amount from 0.5 to 3.00 mmol/L and magnesium in an amount from 0.1 to 0.3 mmol/L. As illustrative, non-limiting examples of the organic salt, there are lactate and bicarbonate, which may be used in an amount from 30 to 60 mmol/L.

As for the osmotic pressure of the composition for peritoneal dialysis in accordance with the present invention, it is set in a range from 300 to 1100 mOsm/l, and preferably in a range from 350 to 1,050 mOsm/l.

In accordance with another aspect, the present invention pertains to a method of conducting peritoneal dialysis by injecting the composition of the present invention into patients with renal failure.

As used herein, the term “patients with renal failure” means all patients who suffer from acute or chronic renal failure, including humans and other mammals, such as mice, rats, rabbits, monkeys, etc.

In an embodiment, a dialysis solution comprising α-keto amino acid according to the present invention is instilled in an amount from about 1.5 to 3.0 liters into the peritoneal cavity via a catheter which is placed in the abdomen, and allowed to dwell for 5 to 6 hrs therein before being drained. This process of filling and draining is conducted three to five times a day.

A better understanding of the present invention may be obtained in light of the following examples which are set forth to illustrate, but are not to be construed to limit the present invention.

### EXAMPLE 1

### Preparation of Dialysate for Peritoneal Dialysis Comprising α-Keto Amino Acid

In the gambrosol trio 40 C solution (Gambro Lundia AB, Sweden) the composition of which is given in Table 1, the commercially available α-keto amino acid complex Ketosteril™ (Fresenius Kabi Deutschland GmbH, Germany) the composition of which is given in Table 2, below, was dissolved in a concentration of 11.55 g/L, so as to afford a composition for peritoneal dialysis comprising α-keto amino acids in accordance with the present invention.

TABLE 1

| Ingredient | Mass (g/L) |
|---|---|
| Sodium Chloride | 5.38 |
| Calcium Chloride Dihydrate | 0.271 |
| Magnesium Chloride Hexahydrate | 0.054 |
| Sodium Lactate | 4.72 |
| pH | 6.6 |

TABLE 2

| Ingredient | Mass (mg) |
|---|---|
| α-keto Isoleucine | 67 |
| α-keto Leucine | 101 |
| α-keto Phenylalanine | 68 |
| α-keto Valine | 86 |
| Hydroxymethionine | 59 |
| L-Lysine | 75 |
| L-Threonine | 53 |
| L-Tryptophan | 23 |
| L-Histidine | 38 |
| L-Tyrosine | 30 |

### EXAMPLE 2

### Analysis of Concentration of Amino Acids and Keto-Amino Acids Using Spectrophotometer

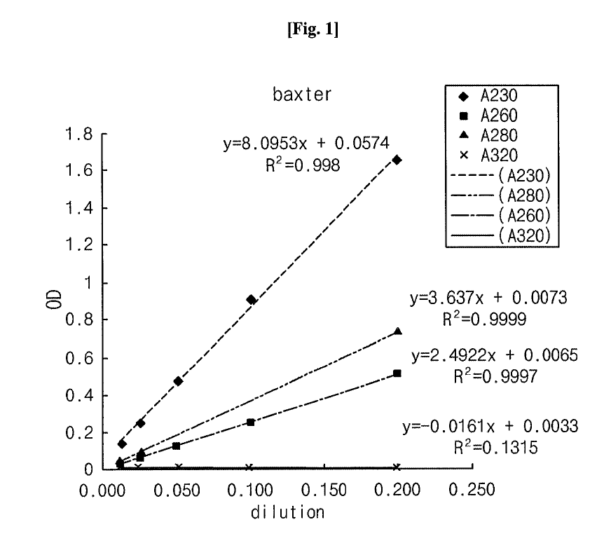

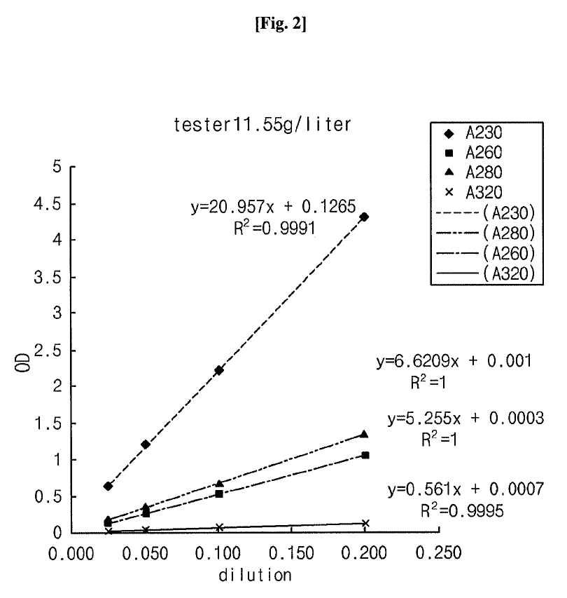

Using a UV spectrophotometer (UV1601, Shimadzu, Japan), the composition comprising α-keto amino acids, prepared in Example 1, was analyzed for amino acid concentration change over time while the Neutril solution comprising amino acids was used a control. The two solutions were measured for absorbance at various wavelengths from 230 to 350 nm in order to examine the diffusion of amino acid derivatives. In this regard, UV spectra were obtained by measuring the absorbance at various wavelengths using a UV spectrophotometer (UV1601, Shimadzu, Japan). Concentrations were determined by comparing absorbance values at 280 nm, the wavelength usually used for the quantitative analysis of amino acids. The results are given in Tables 3 and 4, below, and depicted in respectively corresponding FIGS. 1 and 2.

TABLE 3
Absorbance of Neutril Solution According to Dilution Ratio

| | Dilution Ratio | | | | |
|---|---|---|---|---|---|
| | 0.013 | 0.025 | 0.050 | 0.100 | 0.200 |
| A230 | 0.136 | 0.247 | 0.478 | 0.908 | 1.655 |
| A260 | 0.043 | 0.066 | 0.128 | 0.255 | 0.506 |
| A280 | 0.058 | 0.096 | 0.186 | 0.370 | 0.736 |
| A320 | 0.008 | 0 | 0 | 0.001 | 0.001 |

TABLE 4
Absorbance of Composition for Peritoneal Dialysis Comprising
α-Keto Amino Acid According to Dilution Ratio

Dilution Ratio

| | 0.025 | 0.050 | 0.100 | 0.200 |
|---|---|---|---|---|
| A230 | 0.632 | 1.202 | 2.213 | — |
| A260 | 0.132 | 0.264 | 0.524 | 1.052 |
| A280 | 0.167 | 0.333 | 0.661 | 1.326 |
| A320 | 0.014 | 0.03 | 0.056 | 0.113 |

Amino acids and α-keto amino acids, although showing high molecular similarity to each other, differ from each other in that an α-amino moiety is different from an α-ketone moiety. The two solutions used in the test were estimated to be different in absorbance between two solutes at the same concentration because the compositions of the derivatives differed from one solution to the other solution. Test results indicated that both of the solutions showed a linear relationship between absorbance and concentration within the range used in the test. Hence, the absorbance obtained in the test could be used to determine changes in concentration of the solutions.

### EXAMPLE 3

### Absorption Peak According to Wavelength Band

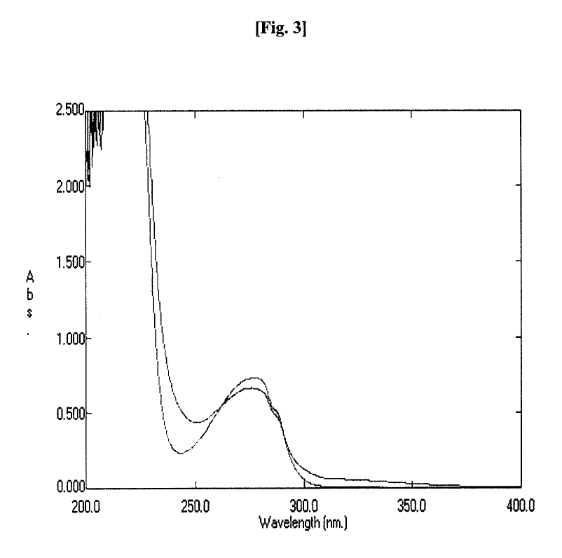

A control (Neutril) and a test group (α-keto amino acid) were examined with respect to absorption peak according to wavelength band. The results are given in Table 5 corresponding to FIG. 3.

TABLE 5
Absorption Peaks of Neutril and α-Keto Amino Acid Solution

| Solution | Wavelength (nm) | Absorption peak |
|---|---|---|
| Control | 276.6 | 0.732 |
| Test | 275.6 | 0.663 |

Absorbance peaks at 280 nm of the two solutions were coincident with each other, indicating that absorbance at 280 nm can be used for the quantitative analysis of the keto acid used as a test solution.

### EXAMPLE 4

### Change in the Mass of Dialysate and the Concentration of α-Keto Amino Acid with Time

100 ml of the composition for peritoneal dialysis comprising α-keto amino acid, prepared in Example 1, was placed in a molecular porous membrane sac which had been heated 30 min in distilled water to remove impurities therefrom. For comparison, 100 ml of Neutril PD-4 (Baxter Healthcare SA, Singapore branch) as a control was placed in another membrane sac. To conduct dialysis, these sacs were floated in 1 liter of the dialysis buffer Hartman's solution (CJ Inc., Korea) in respective baths with a magnetic bar spinning on the bottom. Whenever the dialysis was conducted for 0, 5, 30, 60, 120, and 240 min, 1 ml was sampled from each of the dialysates and the dialysis buffer so as to monitor the mass and concentration thereof.

With dialysis in progress, the solutions inside and outside the dialysis membranes were observed for change in mass and α-keto amino acid concentration. The results are given in Table 6, which shows changes in concentration of the dialysates and the dialysis buffer with time, and in Table 7, which shows changes in mass with time.

TABLE 6
Change in Concentration of Dialysates
and Dialysis Buffer with Time

| | Time (min) | | | | | |
|---|---|---|---|---|---|---|
| Solutions | 0 | 5 | 30 | 60 | 120 | 240 |
| Control Dialysate | 2.698 | 2.252 | 2.139 | 1.927 | 1.313 | 0.991 |
| Control Dialysis Buffer | 0 | 0.007 | 0.034 | 0.059 | 0.089 | 0.137 |
| Test Dialysate | 2.395 | 2.297 | 2.103 | 1.831 | 1.350 | 1.054 |
| Control Dialysis Buffer | 0 | 0.006 | 0.029 | 0.053 | 0.080 | 0.126 |

TABLE 7
Change in Mass of Dialysates with Time

Time (min)

| | 0 | 5 | 30 | 60 | 120 | 240 |
|---|---|---|---|---|---|---|
| Control | 143.1 | 143.2 | 143.9 | 144.4 | 145.4 | 147.2 |
| Test | 143.0 | 143.1 | 143.9 | 144.3 | 145.3 | 147.1 |

The concentrations of amino acid in the dialysates and the dialysis buffer were observed to change with time very similarly between the control and the test group. In particular, the ratio of dialysis buffer concentration to the initial dialysate concentration changed over time in almost the same pattern between the control and the test group, indicating that the motility of amino acids across semi-permeable membranes was almost coincident with that of α-keto amino acids. Also, the two groups were observed to show similar results with regard to the change in mass of dialysate.

As proven in this experiment, the 1.1% α-keto amino acid dialysate has the same dialysis properties and motility across a semi-permeable membrane as the conventional amino acid dialysate.

### EXAMPLE 5

### Safety Assay of Dialysate for Peritoneal Dialysis Comprising α-Keto Amino Acid

5-1. Preparation of Peritoneal Dialysis Solution Comprising α-Keto Amino Acid

In distilled water were dissolved α-keto amino acids of Table 2 (Ketosteril™, Germany) at a concentration of 12.735 g/L, sodium at a concentration of 132 mmol/L, calcium at a concentration of 1.25 mmol/L, magnesium at a concentration of 0.25 mmol/L, lactate at a concentration of 40 mmol/L, and chlorine at a concentration of 105 mmol/L, followed by autoclaving at 120° C. for 20 min to prepare a composition for peritoneal dialysis comprising α-keto amino acids. The composition prepared was measured to have a pH of 6.7 and an osmotic pressure of 366 mOsm/L.

5-2. Preparation of Peritoneal Dialysis Catheter

A silicon tubing (Cole Palmer Instrument Company, Chicago, Ill.) having an inner diameter of 1/16 inches and an outer diameter of ⅛ inches was cut to a length of 12 inches. Polyester cuffs, each about 1 cm wide, were firmly fixed at sites respectively 1 and 3 inches distant from the end thereof with a silastic medical adhesive (Dow Corning Corp., Midland, Mich.), followed by forming 20 pores, each 1 mm in diameter, in the side thereof.

After cutting the end of a 15 gauge needle, its lumen was closed for use as a stopper for the catheter.

5-3. Animal Test

9 male Sprague Dawley mice, each weighing 250-300 g, were etherized. A ventral midline incision of 3 cm lengths was made from the xiphoid in the downward direction before a laparotomy was made about 1.5 cm below the xiphoid to open the peritoneal cavity. The prepared peritoneal dialysis catheter was inserted into the peritoneal cavity, which was then sutured to fix the cuff to the abdominal wall. The external portion of the catheter was drawn through the subcutaneous tunnel to the larynx between the scapulas, and closed with a catheter stopper. The incision, through which the catheter was inserted, was closed with a 9-mm surgical clip.

The mice, into which the peritoneal dialysis catheters were inserted, were divided into three groups of three: one injected with a standard glucose dialysis fluid (Dianeal, Baxter Inc), another injected with a standard amino acid dialysis fluid (Nutrineal, Baxter Inc), and the other injected with the α-keto amino acid dialysis fluid. For all of the groups, the dialysates were injected in an amount of 30 cc twice a day.

During the experiment of 0, 1, 3 and 5 weeks, blood levels of hsCRP, BUN, creatinine, proteins and albumin were analyzed using a calorimetric method and a calorimetric immunoassay (ADVIA 1650, Bayer, U.S.A.) (FIGS. 4 to 8). After dialysis for 5 weeks, the mice were anesthetized with 50 mg/kg of pentothal and underwent a laparotomy to excise the peritoneum. After fixation with 10% neutral-buffered formalin, the peritoneum was treated according to a standard protocol and cut into 5-mm pieces. The peritoneum samples were stained with trichrome and analyzed for fibrosis.

5-4. Experiment Results

(1) Survival

All mice of each group were alive even after dialysis was conducted for five weeks.

(2) Test Indices

There was no significant difference in mean values of blood levels of glucose, BUN, creatinine, α-keto amino acids among the groups, that is, the glucose dialysis fluid group, the amino acid dialysis fluid group, and the α-keto amino acid dialysis fluid group over zero, 1, 3 and 5 weeks (FIGS. 4 to 9). The renal function indices, BUN and creatinine levels were maintained constant in all the groups. Also, the nutrition indices, protein and albumin levels, were constant in all the groups. There was no significant difference in the inflammation index hsCRP among the groups.

The lack of significant difference in either hsCRP or albumin indicates that the α-keto amino acid dialysate for peritoneal dialysis according to the present invention has the same level of safety as standard glucose or amino acid dialysates.

### INDUSTRIAL APPLICABILITY

As described hitherto, the composition for peritoneal dialysis comprising an α-keto amino acid in accordance with the present invention prevents the problems with conventional glucose or amino acid dialysates for peritoneal dialysis, including peritoneal injury, hyperlipidemia, cardiovascular injury, and increase of BUN, and thus allows peritoneal dialysis to be effected with higher safety and efficiency.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced other than as specifically described.

1. A composition for peritoneal dialysis, comprising an α-keto amino acid.

2. The composition as set forth in claim 1, wherein the α-keto amino acid is selected from a group consisting of glycine, alanine, serine, cysteine, aspartic acid, glutamine, glutamic acid, leucine, isoleucine, lysine, hydroxylysine, asparagine, tyrosine, tryptophan, histidine, phenylalanine, cystine, proline, hydroxyproline, threonine, methionine, hydroxymethionine, valine and combinations thereof, each having an α-ketone moiety instead of an α-amino moiety of a standard amino acid.

3. The composition as set forth in claim 2, wherein the α-keto amino acid is selected from a group consisting of leucine, isoleucine, phenylalanine, valine and combinations thereof, each having an α-ketone moiety instead of an α-amino moiety of a standard amino acid.

4. The composition as set forth in claim 1, wherein the α-keto amino acid comprises leucine, isoleucine, phenylalanine and valine, each having an α-ketone moiety instead of an α-amino moiety of a standard amino acid.

5. The composition as set forth in claim 1, wherein the α-keto amino acid is contained in an amount from 8,000 to 40,000 mg/l.

6. The composition as set forth in claim 1, further comprising at least one ingredient selected from a group consisting of an amino acid, glucose and an electrolyte.

7. The composition as set forth in claim 6, wherein the amino acid is selected from a group consisting of glycine, alanine, serine, cysteine, aspartic acid, glutamine, glutamic acid, leucine, isoleucine, lysine, hydroxylysine, asparagine, tyrosine, tryptophan, histidine, phenylalanine, cystine, proline, hydroxyproline, threonine, methionine, hydroxymethionine, valine and combinations thereof.

8. The composition as set forth in claim 7, wherein the amino acid is selected from a group consisting of lysine, threonine, tryptophan, histidine, tyrosine, hydroxymethionine and combinations thereof.

9. The composition as set forth in claim 6, wherein the amino acid is contained in an amount from 280 to 1200 mg/l.

10. The composition as set forth in claim 6, wherein the electrolyte is selected from a group consisting of sodium, potassium, chlorine, calcium, magnesium and combinations thereof.

11. The composition as set forth in claim 10, wherein the electrolyte comprises sodium in an amount from 125 to 140 mmol/L, potassium in an amount from 0 to 4 mmol/L, a chloride ion in an amount from 95 to 115 mmol/L, calcium in an amount from 0.5 to 3.00 mmol/L and magnesium in an amount from 0.1 to 0.3 mmol/L.

12. The composition as set forth in claim 5, further comprising an organic acid salt.

13. The composition as set forth in claim 12, wherein the organic acid salt is selected from a group consisting of lactate, bicarbonate and a combination thereof.

14. The composition as set forth in claim 12, wherein the organic acid salt is contained in an amount from 30 to 60 mmol/L.

15. A method for peritoneal dialysis, comprising administering the composition of claim 1 to a patient with renal failure.
